(19) United States
(12) Patent Application Publication
(10) Pub. No.: US 2024/0189299 A1
(43) Pub. Date: Jun. 13, 2024

(54) ORAL FILM OF CFTR MODULATOR(S)

(71) Applicant: LAURUS LABS LIMITED, Hyderabad (IN)

(72) Inventors: Hemant Bhagwat NARKHEDE, Hyderabad (IN); Srikant PIMPLE, Hyderabad (IN); Siva Ramakrishna RAYALA, Hyderabad (IN); Ramgopal RAO K., Hyderabad (IN); Siva Ramakrishna VELAGA, Hyderabad (IN); Satheesh JOGALA, Hyderabad (IN)

(21) Appl. No.: 18/549,234

(22) PCT Filed: Mar. 8, 2022
(86) PCT No.: PCT/IN2022/050207

§ 371 Date: Sep. 6, 2023

(30) Foreign Application Priority Data
Mar. 9, 2021 (IN) 202141009765

(51) Int. Cl. A61K 31/47; A61K 9/00; A61K 31/404; A61K 31/443; A61K 31/4439; A61K 47/32; A61K 47/38

(52) U.S. Cl. CPC A61K 31/47; A61K 9/006; A61K 31/404; A61K 31/443; A61K 31/4439; A61K 47/32; A61K 47/38

(57) ABSTRACT

The present invention provides oral film(s) of Cystic fibrosis transmembrane conductance regulator (CFTR) modulator(s) or a pharmaceutically acceptable salt thereof. Further the present invention provides composition and process for preparing oral film(s) comprising Cystic fibrosis transmembrane conductance regulator (CFTR) modulator(s) or a pharmaceutically acceptable salt thereof.

### BACKGROUND OF THE INVENTION

### Field of the Invention

The present invention relates to oral film of CFTR modulator(s) or a pharmaceutically acceptable salt thereof. In particular, the present invention also relates to process for preparing oral film of CFTR modulator(s) or a pharmaceutically acceptable salt thereof. More preferably, the present invention relates to oral film compositions comprising CFTR modulator(s) or a pharmaceutically acceptable salt thereof and administering the same to the oral cavity of the humans for treating Cystic fibrosis.

### Description of the Related Art

Cystic fibrosis (CF) is described as an inherited life-threatening disorder that damages the lungs and digestive system. CF is a genetic disorder that affects mostly the lungs but also the pancreas, liver, kidneys, and intestine. CF affects the cells that produce mucus, sweat, and digestive juices. It causes these fluids to become thick and sticky. They then plug up tubes, ducts, and passage ways. Long-term issues of Cystic fibrosis include difficulty in breathing and coughing up mucus as a result of frequent lung infections. Other signs and symptoms include sinus infections, poor growth, fatty stool, clubbing of the fingers and toes, and infertility in males. CF is inherited in an autosomal recessive manner. It is caused by mutations in the cystic fibrosis transmembrane conductance regulator (CFTR) gene that affect the production of the CFTR protein. When the CFTR protein is not made correctly, it affects the balance of salt and fluids inside and outside of the cell. This imbalance leads to thick, sticky mucus in the lungs, pancreas, and other organs.

The cystic fibrosis transmembrane conductance regulator (CFTR) protein helps to maintain the balance of salt and water on many surfaces in the body, such as the surface of the lung. When the protein is not working correctly, chloride—a component of salt—becomes trapped in cells. Without the proper movement of chloride, water cannot hydrate the cellular surface. This leads the mucus covering the cells to become thick and sticky, causing many of the symptoms associated with cystic fibrosis.

The CFTR protein regulates the proper flow of water and chloride in and out of cells lining the lungs and other organs. In people with CF, mutations in the CFTR gene result in either a defective protein being produced or no protein at all. This leads to the buildup of thick, sticky mucus, which can lead to infections in the lungs and damage to the pancreas. It can also lead to problems in other parts of the body.

There are no cures for cystic fibrosis, there are several treatment methods. Recent advances in the treatment of cystic fibrosis have meant that an individual with cystic fibrosis can live a fuller life less encumbered by their condition. Many people with CF are on one or more antibiotics at all times, even when healthy, to prophylactically suppress infection.

Cystic fibrosis transmembrane conductance regulator (CFTR) modulators are designed to correct the malfunctioning protein made by the CFTR gene. Because different mutations cause different defects in the protein, the medications that have been developed so far are effective only in people with specific mutations. In recent years following four CFTR modulators which are currently approved by United States Food and Drug Administration (USFDA) for people with certain CFTR mutations:
Kalydeco® (ivacaftor)
Orkambi® (lumacaftor/ivacaftor)
Symdeko® (tezacaftor/ivacaftor)
Trikafta® (elexacaftor/tezacaftor/ivacaftor)

CFTR modulators intended to modulate the function of the CFTR protein so that it can serve its primary function: to create a channel for chloride (a component of salt) to flow across the cell surface. When proper chloride flow is reestablished, mucus becomes rehydrated inside the lungs and other organs. Although modulators can't yet completely restore proper chloride flow, they can improve the flow enough to relieve symptoms for people with CF.

There are three main types of CFTR modulators:
1. Potentiators
2. Correctors
3. Amplifiers

Potentiators: The CFTR protein is shaped like a tunnel that can be closed by a gate. Potentiators are CFTR modulators that hold the gate open so chloride can flow through the cell membrane.

The drug ivacaftor (Kalydeco®) is a potentiator. This drug can help patients with gating and conduction mutations in CFTR. It also works on residual function and splice mutations where an insufficient amount of normal protein is present. In all these mutations, some CFTR protein reaches the surface of the cell. However, either not enough protein reaches the cell surface, or the protein does not allow enough chloride to flow through. By holding the gate on the CFTR protein open, potentiators allow more chloride to flow through and reduce the symptoms of CF.

Correctors: The next type of CFTR modulator is called a “corrector.” Correctors help the CFTR protein to form the right 3-D shape so that it is able to move—or traffic—to the cell surface.

Nearly 90 percent of people with CF have at least one copy of the F508del mutation, which prevents the CFTR protein from forming the right shape. Corrector drugs help the CFTR protein to form the right shape, traffic to the cell surface, and stay there longer. But, even with correctors, only some of the CFTR protein reaches the cell surface. Additionally, the proteins that do reach the cell surface do not open sufficiently to allow chloride to pass out of the cell.

But, if a corrector(s) is used in combination with a potentiator—such as ivacaftor—to hold the gate on the CFTR protein open, enough chloride can then flow to reduce the symptoms of CF. In the newest modulator on the market, the correctors elexacaftor and tezacaftor were combined with ivacaftor to form Trikafta®, a triple combination that can be used to treat people with CF who have at least one copy of the F508del mutation (regardless of their second mutation). Earlier dual-combination drugs, such as lumacaftor/ivacaftor (Orkambi®) and tezacaftor/ivacaftor (Symdeko®), served a smaller population—people with two copies of the F508del mutation. Symdeko® also can be used to treat people with a single copy of one of 26 specified mutations—regardless of their other mutation.

Amplifiers: The last type of CFTR modulator is called an “amplifier” Amplifiers increase the amount of CFTR protein that the cell makes. Many CFTR mutations produce insufficient CFTR protein. If the cell made more CFTR protein, potentiators and correctors would be able to allow even more chloride to flow across the cell membrane. Amplifiers, which are being developed and tested, are not yet available.

Kalydeco® (ivacaftor) is marketed as 150 mg film-coated tablet and granules for oral administration (sweetened but unflavoured) enclosed in a unit-dose packet containing 25 mg of ivacaftor, 50 mg of ivacaftor or 75 mg of ivacaftor. The granules for oral administration are basically used for paediatric patients aged 4 months to less than 6 years.

Orkambi® (lumacaftor/ivacaftor) is marketed as film-coated tablets in fixed dose combination of lumacaftor 200 mg/ivacaftor 125 mg; lumacaftor 100 mg/ivacaftor 125 mg and granules for oral administration enclosed in a unit-dose packet containing Lumacaftor 100 mg/ivacaftor 125 mg or Lumacaftor 150 mg/ivacaftor 188 mg per packet. The granules for oral administration are basically used for children aged 2 years to less than 6 years.

Symdeko® (tezacaftor/ivacaftor) is marketed as co-package containing tablets that constitute a daily dosage in fixed dose combination of Tezacaftor 50 mg/ivacaftor 75 mg or tezacaftor 100 mg/ivacaftor 150 mg for administration during morning. Besides, co-package also contains daily dosages of Ivacaftor 75 mg or 150 mg tablets for administration during evening.

Trikafta® (elexacaftor/tezacaftor/ivacaftor) is marketed as co-package containing tablets in fixed dose combination of Elexacaftor 100 mg/Tezacaftor 50 mg/Ivacaftor 75 mg or Elexacaftor 50 mg/Tezacaftor 25 mg/Ivacaftor 37.5 mg for morning administration. Besides, co-package also contains daily dosage of Ivacaftor 75 mg or 150 mg tablets for evening administration.

Following patents/publications discloses various formulations of CFTR modulators:

U.S. Pat. Nos. 8,410,274 and 8,754,224 discloses solid dispersions comprising amorphous Ivacaftor prepared by spray drying with the aid of carriers like water soluble polymers (hydroxypropylmethylcellulose acetate succinate) and surfactants (Sodium lauryl sulfate) and further processing the said solid dispersion into a tablet composition was disclosed in U.S. Pat. No. 10,646,481.

U.S. Pat. Nos. 7,553,855 and 8,076,357 discloses Ivacaftor liquid composition containing liquid polyethylene glycol and polyvinyl pyrrolidone and process for preparing the same.

U.S. Pat. Nos. 8,883,206 and 10,272,046 discloses pharmaceutical composition containing solid dispersions of Ivacaftor encompassed with the carriers like water soluble polymers (hydroxypropylmethylcellulose acetate succinate) and surfactant; Further, the solid dispersions were formulated into powders, granules and mini-tablets by using diluent/filler, sweetener, disintegrant, glidant and lubricant for administering in pediatric patients.

U.S. Pat. Nos. 9,216,969, 8,653,103, 9,192,606, 10,231,932 and application 20200338063 disclose pharmaceutical compositions, preferably tablets composition comprising crystalline lumacaftor and solid dispersion of amorphous Ivacaftor and manufacturing process.

U.S. Pat. Nos. 9,012,496, 10,058,546, 10,081,621 and 10,206,877 discloses solid dispersions of amorphous Tezacaftor prepared by spray drying with the aid of carriers like hydroxypropylmethylcellulose (HPMC) and hydroxypropyl methylcellulose acetate succinate (HPMCAS) and further discloses pharmaceutical composition comprising the said amorphous dispersion of tezacaftor either alone or in combination with another solid dispersion comprising amorphous ivacaftor.

U.S. Pat. No. 11,179,367 disclose pharmaceutical compositions, preferably tablet composition comprising 1) crystalline Elexacaftor, 2) solid dispersion of amorphous Ivacaftor and 3) solid dispersion of amorphous Tezacaftor and tablet manufacturing.

U.S. Pat. Nos. 10,206,915 and 10,376,501 discloses complexes of ivacaftor and Lumacaftor. Specification discloses several complexing agents. The complex shows improved physicochemical characteristics and enhanced biological performance compared to KALYDECO® and ORKAMBI® respectively.

Pediatric CF patients may require administration of pharmaceutical compositions in a dosage form that facilitates swallowing or that may be easily mixed with easily digested foods. The use of crushed tablets in the administration of pharmaceutical compositions to children has often presented problems in administration and dosing. Administering crushed tablet formulations to children, can lead to absorption problems, fragments that are either too difficult to swallow or fail to solubilize in the food and remain undigested resulting in therapeutic failure, or dosage inaccuracies. Such dosing inaccuracies are particularly prevalent when the person administering the dose is inexperienced and when the dose is small, as in those used to treat pediatric patients. Dosage errors involving CF pharmaceutical active agents therefore become critical in pediatric populations, particularly considering that pharmaceutical CF active agents are administered in low doses (e.g. less than 100 mg or less than 50 mg per unit dose). These dosing inaccuracies become critical in pediatric patients having a low threshold for dose deviation.

Currently, CFTR modulators were available only in tablet and granule dosage forms for oral administration. The granule dosage form is specifically used for paediatric patients available in the market are Kalydeco® and Orkambi®. Granules for oral administration of Kalydeco® and Orkambi® need to be mixed with one teaspoon (5 mL) of age-appropriate soft food or liquid and completely consumed to ensure delivery of the entire dose. Once mixed, the product has been shown to be stable for one hour, and therefore should be consumed during this period. Some examples of appropriate soft foods or liquids may include puréed fruits or vegetables, yogurt, applesauce, water, breast milk, infant formula, milk, or juice. The disadvantage of these granules is, it needs to be administered along with liquid or soft food, where children may show reluctance or dislike to take these granules mixed food with altered taste and smell than the original and the perception of such prepared dosage forms may lead to develop permanent reluctance towards those nutritional foods even served without the CFTR modulator granules. One more disadvantage of granules for oral administration is after mixing the dosage form if the patient did not consume after 1 hour of preparation the dosage form needs to be discarded. The dose will get wasted and more importantly the patient may skip the dose.

If the patient begins taking CFTR modulator(s), then it is an everyday medication regimen continued throughout life. Hence, the dosage forms especially for pediatric cystic fibrosis patients should be more compliance for administering, palatable and show less dosing errors etc.

To overcome problems of tablets and granules for oral administration, difficulty of swallowing by pediatric/geriatric patients, the present inventors have identified simple solution by developing oral films, which can be administered simply by keeping the film on the tongue, the film disintegrates in less than five minutes. The main advantage of the film over tablets/granules for oral administration is there is no need of soft food or liquid required for dosage form administration, no extemporaneous preparation of the dosage form, no dosing errors, easy administration to all age group of patients etc.

### BRIEF SUMMARY OF THE INVENTION

In one embodiment, the present invention provides oral film of CFTR modulator(s) and its pharmaceutically acceptable salts thereof.

In another embodiment, the present invention provides composition and process for preparing oral film of CFTR modulator(s) and its pharmaceutically acceptable salts thereof.

In an embodiment, the present invention provides composition of oral film of CFTR modulator(s) and its pharmaceutically acceptable salts thereof, wherein the composition comprises at least one film forming polymer, plasticizer, optionally carrier(s) and other pharmaceutically acceptable excipients.

In one embodiment, the present invention provides composition of oral film of CFTR modulator(s) and its pharmaceutically acceptable salts, wherein the composition comprises a solid dispersion(s) of CFTR modulator(s), optionally another CFTR modulator(s), at least one film forming polymer, plasticizer and other pharmaceutically acceptable excipients.

In another embodiment, the present invention provides process for preparing oral film comprising CFTR modulator(s) and its pharmaceutically acceptable salts thereof, wherein the process may be selected from solvent casting, hot melt extrusion or printing technology.

In another embodiment, the present invention provides oral film of CFTR modulator(s) and its pharmaceutically acceptable salts thereof, where in the oral film comprises combination of two or more CFTR modulator(s) and their pharmaceutically acceptable salts thereof.

### DETAILED DESCRIPTION OF THE INVENTION

References in the specification to “one embodiment,” “an embodiment,” “another embodiment,” “a preferred embodiment,” “one aspect,” “another aspect,” “preferred aspect”, “further aspect” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is considered that it is within the knowledge of one of ordinary skill in the art to affect such feature, structure, or characteristic in connection with other embodiments whether or not explicitly described.

The term “CFTR modulator” or “CFTR modulator drug” according to the present invention refers to Cystic fibrosis transmembrane conductance regulator modulator drugs or therapies for treating cystic fibrosis.

As per United States Pharmacopoeia, Films are classified by the site of application. Accordingly, “Oral film” can be formulated to deliver medication to the mouth such as oral hygiene products or to deliver medication to the gastrointestinal tract for absorption. “Buccal films” and “sublingual films” are formulated to facilitate absorption through the proximal mucosal membranes avoiding first pass metabolism or degradation in the gastrointestinal tract and providing a quick onset of action.

“Oral film” according to the present invention may also referred as “oral thin film”, “OTF”, “ODF”, “oral drug strip”, “oral strip”, “oral disintegrating film” or “oral dissolving film”.

The oral films, according to the present invention preferably disintegrate within about five minutes and more preferably within about 180 seconds upon contact with aqueous media or saliva in oral cavity.

An oral film according to the present invention is “non-mucoadhesive” in nature, means that the dosage form is not designed for administration of the active pharmaceutical agent through the oral mucosa i.e. the dosage form is not designed to adhere to the mucosal surfaces of the buccal cavity as an intact film or disintegrated film residue. In particular, the invention provides a non-mucoadhesive orally disintegrating film, able to disintegrate upon contact with aqueous media or saliva in oral cavity within about five minutes and more preferably within about 180 seconds.

The term “film” according to the present invention includes films, sheets and wafers, in any size and shape, including rectangular, square, or other desired shape. The films described herein may be any desired thickness and size such that it may be placed into the oral cavity of the user. The films may have a thickness of from about 20 microns to about 300 microns. Films may be in a single layer or they may be multi-layered, such as laminated or co-extruded films.

The term “disintegrating” according to present invention is defined as a state in which any residue of the oral film remaining on the screen of the test apparatus known in the art, or in the mouth, is a soft mass having no palpably film core. The disintegration test does not imply complete solution of dosage unit or even of its active constituent, although a dissolved dosage unit would typically be completely disintegrated.

The term “dissolution” according to present invention is defined by the amount of active agent released from the oral film after oral administration or by in-vitro testing known in the art. An in-vitro dissolution test is to evaluate the performance of the product by measuring the amount of active agent dissolved in the dissolution medium. Standardized apparatus known in the art for in-vitro dissolution testing are: USP type I apparatus (basket), USP type II apparatus (Paddle), USP type V apparatus (Paddle over disc).

In one aspect the present invention provides oral film of CFTR modulator(s) and its pharmaceutically acceptable salt thereof.

In another aspect the present invention provides composition and process for preparing oral film of CFTR modulator(s) and its pharmaceutically acceptable salt thereof.

CFTR modulator(s) according to the present invention are selected from ivacaftor, lumacaftor, tezacaftor, elexacaftor, or any combination and its pharmaceutically acceptable salt.

CFTR modulator(s) according to the present invention are selected from the combination of two or more drugs like Ivacaftor/Lumacaftor, Ivacaftor/tezacaftor, Ivacaftor/tezacaftor/elexacaftor or any other combinations and their pharmaceutically acceptable salts thereof.

Pharmaceutically acceptable salt of CFTR modulator(s) according to the present invention is selected from sodium, potassium, magnesium, hydrochloride, calcium, hydrobromide, phosphate, maleate, besylate, fumarate, sulphate etc.

CFTR modulator and its pharmaceutically acceptable salt or combinations of CFTR modulators and its pharmaceutically acceptable salt according to the present invention may be present in an amount of about 5% to about 75% by weight based on total weight of the composition.

In one preferred aspect, the present invention provides composition of oral film of CFTR modulator(s) and its pharmaceutically acceptable salt thereof, wherein the composition comprises at least one film forming polymer, plasticizer, optionally carrier(s) and other pharmaceutically acceptable excipients.

According to the present invention, other pharmaceutically acceptable excipients are selected from the group comprising of suspending/thickening agents, fillers/bulking agents, disintegrating agents, stabilizers, surfactants, sweetening agents, taste masking agents, anti-foaming agents, flavoring agents and coloring agents and combinations thereof.

In one aspect, the present invention provides process for preparing oral film of CFTR modulator(s), wherein the process may be selected from solvent casting, hot melt extrusion or printing technology.

In one aspect, the present invention provides composition of oral film of CFTR modulator(s) and its pharmaceutically acceptable salt thereof, wherein the composition comprises a solid dispersion(s) of CFTR modulator(s), optionally another CFTR modulator(s), at least one film forming polymer, plasticizer and other pharmaceutically acceptable excipients.

In a further aspect, the present invention provides oral film comprising solid dispersion(s) of CFTR modulator(s), wherein the solid dispersion comprises at least one CFTR modulator, carrier(s) and optionally surfactant.

In a further aspect, the process of preparing oral film of the present invention includes process for preparing solid dispersion of CFTR modulator, wherein the process for preparing solid dispersion may be selected from solvent evaporation, freeze drying, co-precipitation, hot melt extrusion or spray drying.

In one aspect, the present invention provides oral film of Ivacaftor and its pharmaceutically acceptable salt thereof.

In one aspect, the present invention provides composition of oral film of Ivacaftor and its pharmaceutically acceptable salt thereof, wherein the composition comprises at least one film forming polymer, plasticizer, optionally carrier(s) and other pharmaceutically acceptable excipients.

In another aspect, the present invention provides process for preparing oral film of Ivacaftor and its pharmaceutically acceptable salt thereof, wherein the process may be selected from solvent casting, hot melt extrusion or printing technology.

In one aspect, the present invention provides composition of oral film of Ivacaftor and its pharmaceutically acceptable salt thereof, wherein the composition comprises solid dispersion of Ivacaftor, at least one film forming polymer, plasticizer and other pharmaceutically acceptable excipients.

In a further aspect, the present invention provides oral film comprising solid dispersion of Ivacaftor and its pharmaceutically acceptable salt thereof, wherein the solid dispersion comprises Ivacaftor, carrier(s) and optionally surfactant.

In one aspect, the present invention provides oral film of Lumacaftor and its pharmaceutically acceptable salt thereof.

In one aspect, the present invention provides composition of oral film of Lumacaftor and its pharmaceutically acceptable salt thereof, wherein the composition comprises at least one film forming polymer, plasticizer, optionally carrier(s) and other pharmaceutically acceptable excipients.

In another aspect, the present invention provides process for preparing oral film of Lumacaftor and its pharmaceutically acceptable salt thereof, wherein the process may be selected from solvent casting, hot melt extrusion or printing technology.

In one aspect, the present invention provides oral film of Tezacaftor and its pharmaceutically acceptable salt thereof.

In one aspect, the present invention provides composition of oral film of Tezacaftor and its pharmaceutically acceptable salt thereof, wherein the composition comprises at least one film forming polymer, plasticizer, optionally carrier(s) and other pharmaceutically acceptable excipients.

In another aspect, the present invention provides process for preparing oral film of Tezacaftor and its pharmaceutically acceptable salt thereof, wherein the process may be selected from solvent casting, hot melt extrusion or printing technology.

In one aspect, the present invention provides composition of oral film of Tezacaftor and its pharmaceutically acceptable salt thereof, wherein the composition comprises solid dispersion of Tezacaftor, at least one film forming polymer, plasticizer and other pharmaceutically acceptable excipients.

In a further aspect, the present invention provides oral film comprising solid dispersion of Tezacaftor, wherein the solid dispersion comprises Tezacaftor, carrier(s) and optionally surfactant.

In one aspect, the present invention provides oral film of Elexacaftor and its pharmaceutically acceptable salt thereof.

In one aspect, the present invention provides composition of oral film of Elexacaftor and its pharmaceutically acceptable salt thereof, wherein the composition comprises at least one film forming polymer, plasticizer, optionally carrier(s) and other pharmaceutically acceptable excipients.

In another aspect, the present invention provides process for preparing oral film of Elexacaftor and its pharmaceutically acceptable salt thereof, wherein the process may be selected from solvent casting, hot melt extrusion or printing technology.

In one aspect, the present invention provides oral film of Ivacaftor/Lumacaftor combination and their pharmaceutically acceptable salt thereof.

In one aspect, the present invention provides composition of oral film of Ivacaftor/Lumacaftor combination and its pharmaceutically acceptable salt thereof, wherein the composition comprises at least one film forming polymer, plasticizer, optionally carrier(s) and other pharmaceutically acceptable excipients.

In another aspect, the present invention provides process for preparing oral film of Ivacaftor/Lumacaftor combination and its pharmaceutically acceptable salt thereof, wherein the process may be selected from solvent casting, hot melt extrusion or printing technology.

In one aspect, the present invention provides composition of oral film of Ivacaftor/Lumacaftor combination and its pharmaceutically acceptable salt thereof, wherein the composition comprises solid dispersion of Ivacaftor, Lumacaftor, at least one film forming polymer, plasticizer and other pharmaceutically acceptable excipients.

In one aspect, the present invention provides oral film of Ivacaftor/Tezacaftor combination and its pharmaceutically acceptable salt thereof.

In one aspect, the present invention provides composition of oral film of Ivacaftor/Tezacaftor combination and its pharmaceutically acceptable salt thereof, wherein the composition comprises at least one film forming polymer, plasticizer, optionally carrier(s) and other pharmaceutically acceptable excipients.

In another aspect, the present invention provides process for preparing oral film of Ivacaftor/Tezacaftor combination and its pharmaceutically acceptable salt thereof, wherein the process may be selected from solvent casting, hot melt extrusion or printing technology.

In one aspect, the present invention provides composition of oral film of Ivacaftor/Tezacaftor combination and its pharmaceutically acceptable salt thereof, wherein the composition comprises solid dispersion of Ivacaftor, solid dispersion of Tezacaftor, at least one film forming polymer, plasticizer and other pharmaceutically acceptable excipients.

In one aspect, the present invention provides composition of oral film of Ivacaftor/Tezacaftor combination and its pharmaceutically acceptable salt thereof, wherein the composition comprises solid dispersion of Ivacaftor and Tezacaftor, at least one film forming polymer, plasticizer and other pharmaceutically acceptable excipients.

In one aspect, the present invention provides oral film of Ivacaftor/Tezacaftor/Elexacaftor combination and its pharmaceutically acceptable salt thereof.

In one aspect, the present invention provides composition of oral film of Ivacaftor/Tezacaftor/Elexacaftor combination and its pharmaceutically acceptable salt thereof, wherein the composition comprises at least one film forming polymer, plasticizer, optionally carrier(s) and other pharmaceutically acceptable excipients.

In another aspect, the present invention provides process for preparing oral film of Ivacaftor/Tezacaftor/Elexacaftor combination and its pharmaceutically acceptable salt thereof. wherein the process may be selected from solvent casting, hot melt extrusion or printing technology.

In one aspect, the present invention provides composition of oral film of Ivacaftor/Tezacaftor/Elexacaftor combination and its pharmaceutically acceptable salt thereof, wherein the composition comprises a solid dispersion of Ivacaftor, a solid dispersion of Tezacaftor, Elexacaftor, at least one film forming polymer, plasticizer and other pharmaceutically acceptable excipients.

In one aspect, the present invention provides composition of oral film of Ivacaftor/Tezacaftor/Elexacaftor combination and its pharmaceutically acceptable salt thereof, wherein the composition comprises a solid dispersion of Ivacaftor and Tezacaftor, Elexacaftor, at least one film forming polymer, plasticizer and other pharmaceutically acceptable excipients.

Film forming agents used in the present invention may be selected from hydrophilic or hydrophobic polymers. Suitable non-limiting hydrophilic film forming polymers according to the present invention are selected from hydroxypropyl methylcellulose, hydroxyethyl cellulose, hydroxypropyl cellulose, sodium carboxy methylcellulose, hydroxypropylmethylcellulose acetate succinate, polyvinyl pyrrolidone or povidone, copovidone, polydextrose, polyvinyl alcohol, polyvinyl acetate, polyethylene oxide, pullulan, sodium alginate, propylene glycol alginate, polyacrylic acid, copolymers of acrylic acid, carboxyvinyl copolymers, modified starch, gelatin, pectin, hydroxypropylethylcellulose, polyoxyethylene stearates, poly-epsilon caprolactone, polyglycolized glycerides, cyclodextrins, carrageenan, galactomannans, polymerized rosin and combinations thereof.

Suitable non-limiting hydrophobic film forming polymers according to the present invention are selected from ethyl cellulose, cellulose acetate, butyl cellulose, polymethacrylates (commercially available as Eudragit S 100, Eudragit RL, Eudragit RS, Eudragit E etc.), shellac, stearic acid, glyceryl behenate, palmitic acid and combinations thereof.

Film forming polymers according to the present invention may be present in an amount of about 1% to about 60% by weight based on total weight of the composition.

The term “plasticizers” according to the present invention are responsible for mechanical properties of the film, such as tensile strength and decrease the fragility of film by decreasing the glass transition temperature of polymer. Suitable non-limiting plasticizers according to the present invention are selected from polyethylene glycol, propylene glycol, polyethylene-propylene glycol, glycerol/glycerin, glycerol monoacetate, diacetate or triacetate, triacetin, polysorbate, cetyl alcohol, sorbitol, sodium diethylsulfosuccinate, triethyl citrate, tributyl citrate, phthalate derivatives such as dimethyl, diethyl and dibutyl phthalate and combinations thereof. Plasticizers according to the present invention may be present in an amount of about 1% to about 15% by weight based on total weight of the composition.

Suitable carriers according to the present invention are selected from povidone, copovidone, polyvinyl alcohol, cellulose derivatives including hydroxypropylmethylcellulose acetate succinate, polyvinyl caprolactam-polyvinyl acetate-polyethylene glycol graft co-polymer, hydroxypropyl methylcellulose, hydroxypropyl cellulose, sodium carboxymethylcellulose, hydroxyethyl cellulose, hydroxypropylmethylcellulose phthalate, polymethacrylic polymers (commercially available as Eudragit RS; Eudragit RL, Eudragit NE, Eudragit E), polyethylene glycols, polyoxyethylene oxide, poloxamers, polyoxyethylene stearates, poly-epsilon caprolactone, polyglycolized glycerides, pectins, cyclodextrins, galactomannans, dextrin, mannitol, alginates, carragenan, xanthan gum and mixtures thereof. Carrier(s) according to the present invention may be present in an amount from 0% to about 50% by weight based on total weight of the composition.

Suspending/thickening agents according to the present invention are responsible for improving the viscosity of the drug and excipient dispersion preparation and consistency of the oral film. Suitable non-limiting suspending/thickening agents according to the present invention are selected from the group comprising of maltodextrin, natural gums like xanthan gum, carrageen, locust bean gum, cellulose derivatives, dextrin, modified starch and combinations thereof.

Suitable non-limiting filler/bulking agents according to the present invention are selected from Starches, anhydrous lactose, mannitol, Maltodextrin, dicalcium phosphate, calcium sulfate, cellulose, kaolin, sodium chloride, sorbitol, sucrose and combinations thereof.

Disintegrating agents according to the present invention are responsible for quick disintegration of oral film upon contact with aqueous media or saliva in oral cavity. Suitable non-limiting disintegrating agents according to the present invention are selected from starch, modified starches, colloidal silicon dioxide, microcrystalline cellulose, crospovidone, Sodium Starch glycolate, croscarmellose sodium and combinations thereof.

“Sweetening agent or Sweetener” according to the present invention enhances the palatable/taste and pleasurable factor, which makes the film relatively acceptable or agreeable to the patient. Suitable non-limiting sweeteners according to the present invention are selected from glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Ammonium Glycyrrhizinate, Stevia rebaudiana (Stevioside); chloro derivatives of sucrose such as sucralose; sugar alcohols such as sorbitol, mannitol, maltodextrin, xylitol, and the like, hydrogenated starch hydrolysates and the synthetic sweetener 3,6-dihydro-6-methyl-1-1-1,2,3-oxathiazin-4-one-2,2-dioxide, particularly the potassium salt (Acesulfame-K), and sodium and calcium salts thereof, natural intensive sweeteners, protein based sweeteners such as Thaumatoccous danielli (Thaumatin I and II) and combinations thereof.

“Taste masking agents” according to the present invention are responsible for blocking or diminishing the bitter taste of drug substance or excipients present in the film. Suitable non-limiting taste masking agents according to the present invention are selected from ion exchange resins, cyclodextrins and its derivatives, ready made available mixtures of taste mask enhancers and combinations thereof.

Stabilizers according to the present invention are responsible improving the shelf life by preventing the degradation of active agents. Suitable non-limiting stabilizers according to the present invention are selected from the group comprising of tocopherol, ascorbic acid, citric acid, sodium bisulfite, sodium metabisulfite, propyl gallate, potassium metabisulfite, butylated hydroxytoluene, butylated hydroxyanisole, EDTA, xanthan gum and combinations thereof.

Suitable non-limiting surfactants according to the present invention are selected from cetyl alcohol, sodium lauryl sulfate, Polyoxyl 40, Polyoxyl 40 hydrogenated castor oil, Polyoxyl 15 hydroxystearate, Polysorbate 80, Spans®, Tweens®, polyoxyethylene sorbitan fatty acid ester, Ethoxylated oils, poloxamers 407 and combinations thereof.

Suitable anti-foaming agents according to the present invention are selected from simethicone, dimethicone or any agent that removes air bubbles/entrapped air/void from film-forming compositions.

“Flavors” used in the present invention are responsible for masking the bitter or nauseating taste of incorporated drug. Suitable non-limiting flavoring agents according to the present invention are selected from natural and synthetic flavoring liquids such agents includes volatile oils, synthetic flavor oils, flavoring aromatics, oils, liquids, oleoresins or extracts derived from plants, leaves, flowers, fruits, stems and combinations thereof. Mint oils like peppermint, cocoa, and citrus oils such as lemon, orange, grape, lime and grapefruit and fruit essences including apple, pear, peach, grape, strawberry, raspberry, cherry, plum, pineapple, apricot, dairy products, natural extracts of meat or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), and combinations thereof.

Suitable coloring agents according to the present invention are selected from food, drug and cosmetic colors (FD&C), drug and cosmetic colors (D&C), or external drug and cosmetic colors (Ext. D&C). These colors are dyes, their corresponding lakes, certain natural and derived colorants. Azo dyes, organic or inorganic pigments, or coloring agents of natural origin and combinations thereof.

In another aspect, some of the excipients according to the present invention used in the oral film may have one or more functions i.e. an excipient used in oral film of the present invention may have multi-function like some of the carriers used in the present invention may also act as film forming agent or plasticizer; starch and modified starch used in the present invention may also act as a bulking agent and/or disintegrating agent; Maltodextrin used in the present invention may act as a sweetener and/or suspending/thickening agent and/or filler/bulking agent; Mannitol used in the present invention may act as a carrier and/or sweetener and/or as a filler/bulking agent; cellulose derivatives used in the present invention may act as a film forming polymer and/or carrier and/or suspending/thickening agent. Hence, a skilled person should not construe the limit of an excipient stated or illustrated in any aspect or embodiment or an example of oral film in the present invention to a single function.

In one aspect, the present invention provides composition of oral film of CFTR modulator(s) and its pharmaceutically acceptable salt thereof, wherein the composition comprises:
i. from about 5% to about 75% by weight of CFTR modulator or combinations of CFTR modulators and its pharmaceutically acceptable salts;
ii. from about 1% to about 60% by weight of at least one film forming polymer;
iii. from about 1% to about 15% by weight of plasticizer;
iv. from 0% to about 50% by weight of carrier(s); and
v. other pharmaceutically acceptable excipients.
   wherein the percentage by weight is relative to the total weight of the composition.

In another aspect, the present invention provides composition of oral film of CFTR modulator(s) and its pharmaceutically acceptable salt thereof, wherein the composition comprises:
i. from about 20% to about 95% by weight of solid dispersion(s) of CFTR modulator(s) and its pharmaceutically acceptable salts;
ii. from about 1% to about 60% by weight of at least one film forming polymer;
iii. from about 1% to about 15% by weight of plasticizer;
iv. other pharmaceutically acceptable excipients.
   wherein the percentage by weight is relative to the total weight of the composition.

Solvents used according to the present invention for preparing oral film of CFTR modulator(s) and its pharmaceutically acceptable salts may be selected from water, methanol, ethanol, dichloromethane, isopropanol, acetone, methylene chloride, methyl ethyl ketone or any combination thereof.

In one aspect, the present invention provides a kit comprising more than one oral film of CFTR modulator(s) in a pack.

In one aspect, the present invention provides a kit comprising: a) oral film(s) of Ivacaftor and b) oral film(s) of Lumacaftor.

In one aspect, the present invention provides a kit comprising more than one oral film of Ivacaftor/Lumacaftor combination.

In one aspect, the present invention provides a kit comprising: a) oral film(s) of Ivacaftor and b) oral film(s) of Tezacaftor.

In another aspect, the present invention provides a kit comprising: a) oral film(s) of Ivacaftor and b) oral film(s) of Ivacaftor/Tezacaftor combination.

In one aspect, the present invention provides a kit comprising: a) oral film(s) of Ivacaftor, b) oral film(s) of Tezacaftor and c) oral film(s) of Elexacaftor.

In another aspect, the present invention provides a kit comprising: a) oral film(s) of Ivacaftor and b) oral film(s) of Ivacaftor/Tezacaftor/Elexacaftor combination.

In one aspect, the present invention provides oral films of CFTR modulator and its pharmaceutically acceptable salts and combinations of CFTR modulators thereof for the treatment of Cystic fibrosis in humans of all age groups, preferably in pediatric patients.

In a further aspect, the present invention provides oral film of CFTR modulator(s) and its pharmaceutically acceptable salts, wherein the amount or dose of each CFTR modulator ranges from about 10 mg to about 250 mg per film.

The said invention is further illustrated by following non-limiting examples: which are set forth to aid in understanding of the invention, and are not intended, and should not be construed, to limit in any way the invention set forth in the claims that follow thereafter. A person skilled in the art will readily recognize the various modifications and variations that may be performed without altering the scope of the present invention. Such modifications and variations are encompassed within the scope of the invention and the examples do not in any way limit the scope of the invention.

The invention is further illustrated by following non-limiting examples:

### Example 1: Oral Film of Ivacaftor 25 mg

TABLE 1

| Sr. No | Ingredients | mg/film |
|---|---|---|
| 1 | Ivacaftor | 25 |
| 2 | Hydroxypropylmethyl cellulose acetate succinate | 6 |
| 3 | Sodium Lauryl Sulfate | 0.20 |
| 4 | Methyl Ethyl Ketone | q.s. |
| 5 | Purified water | q.s. |
| 6 | Hydroxypropyl Methyl Cellulose | 46 |
| 7 | Purified water | q.s |
| 8 | Polyvinyl pyrrolidone | 4 |
| 9 | Mannitol | 6.80 |
| 10 | Sucralose | 2 |
| 11 | Glycerine | 10 |
| 12 | Crospovidone | 10 |
| 13 | Talc | 4 |
| 14 | Orange Juice Flavor | 8 |
| 15 | Peppermint Flavor | 8 |

| | |
|---|---|
| Film weight | 130 |

q.s: quantity sufficient

### Manufacturing Process:

### Stage-A: Preparation of Ivacaftor Dispersion

1. Add Methyl Ethyl Ketone to purified water under stirring and continue stirring for 5 min.
2. Add Hydroxypropylmethyl cellulose acetate succinate to step 1 under stirring followed by sodium lauryl sulfate under stirring and continue stirring till to get homogenous solution.
3. Add Ivacaftor to above solution of step 2 under stirring and continue stirring till to get homogenous dispersion.

### Stage-B: Preparation of Polymer Solution

4. In a separate vessel, add Hydroxypropyl methylcellulose to purified water under stirring and continue stirring to get homogenous solution.
5. Add Polyvinyl pyrrolidone to step 4 under stirring followed by Mannitol, Sucralose, Crospovidone, talc under stirring and continue stirring till to get homogenous solution.

### Stage-C:

Mix Ivacaftor Dispersion of Stage A and Polymer Solution of Stage B to Get a Uniform Dispersion by Either Stirring or by Homogenization.
6. Add Orange juice flavour, Peppermint flavour and glycerine to the dispersion of stage C under stirring and continue stirring till a homogenous dispersion is obtained.
7. If any foam is observed, de-aerate the dispersion by applying vacuum under slow stirring.
8. The dispersion of Step 6/7 is casted using a film casting machine where the dispersion is spreaded on a backing film with a knife to form a thin wet film which passed through a drying zone.
9. The dried film is cut into a desired size by using slitter.
10. Pack the film into suitable pouches/sachets.

### Example 2: Oral Film of Lumacaftor 50 mg

TABLE 2

| Sr. No | Ingredients | mg/film |
|---|---|---|
| 1 | Lumacaftor | 50 |
| 2 | Methyl Ethyl Ketone | q.s |
| 3 | Purified Water | q.s |
| 4 | Polyvinyl pyrrolidone | 10 |
| 5 | Sodium Lauryl Sulfate | 0.50 |
| 6 | Mannitol | 15 |
| 7 | Sucralose | 2.50 |
| 8 | Glycerine | 7.50 |
| 9 | Crospovidone | 10 |
| 10 | Talc | 5 |
| 11 | Orange Juice Flavor | 4 |
| 12 | Peppermint Flavor | 4 |
| 13 | Hydroxypropyl Methyl Cellulose | 115 |
| 14 | Purified Water | q.s |

| | |
|---|---|
| Film weight | 223.50 |

q.s: quantity sufficient

### Manufacturing Process:

### Stage A: Preparation of Lumacaftor Dispersion

1. Add Methyl Ethyl Ketone to purified water under stirring and continue stirring for 5 min.
2. Add Polyvinyl pyrrolidone to step 1 under stirring followed by sodium lauryl sulfate, mannitol, sucralose, crospovidone, talc under stirring and continue stirring till to get homogenous dispersion.
3. Add Lumacaftor to above solution of step 2 under stirring and continue stirring till to get homogenous dispersion.

### Stage-B: Preparation of Polymer Solution

In a separate vessel, add Hydroxypropyl methylcellulose to purified water under stirring and continue stirring to get homogenous solution

### Stage-C:

Mix Lumacaftor Dispersion of Stage A and Polymer Solution of Stage B Under Stirring and Continue Stirring Till a Homogenous Dispersion is Obtained.
4. Add orange juice flavor, peppermint flavor and glycerine to the dispersion of stage C under stirring and continue stirring till a homogenous dispersion is obtained.
5. Further, follow the steps 7 to 10 described in Example 1 to obtain Lumacaftor oral film.

### Example 3: Oral Film of Ivacaftor 125 mg/Lumacaftor 100 mg

TABLE 3

| Sr. No | Ingredients | mg/film |
|---|---|---|
| 1 | Ivacaftor | 125 |
| 2 | Hydroxypropylmethyl cellulose acetate succinate | 30 |
| 3 | Sodium Lauryl Sulfate | 1 |
| 4 | Methyl Ethyl Ketone | q.s |
| 5 | Purified water | q.s |
| 6 | Lumacaftor | 100 |
| 7 | Hydroxypropyl Methyl Cellulose | 230 |
| 8 | Polyvinylpyrrolidone | 20 |
| 9 | Sodium Lauryl Sulfate | 1 |
| 10 | Mannitol | 30 |
| 11 | Sucralose | 5 |
| 12 | Glycerine | 15 |
| 13 | Crospovidone | 15 |
| 14 | Talc | 6 |
| 15 | Orange Juice Flavor | 8 |
| 16 | Peppermint Flavor | 8 |
| 17 | Methyl Ethyl Ketone | q.s |
| 18 | Purified Water | q.s |

| | |
|---|---|
| Film weight | 594 |

q.s: quantity sufficient

### Manufacturing Process:

### Stage-A: Preparation of Ivacaftor Dispersion:

Ivacaftor dispersion is obtained according the process described in stage A of Example 1.

### Stage-B: Preparation of Lumacaftor Dispersion:

1. In a separate vessel, add Methyl Ethyl Ketone to purified water under stirring and continue stirring for 5 min.
2. Add Hydroxypropyl Methyl Cellulose to above Step 1 under stirring followed by Polyvinyl pyrrolidone, sodium lauryl sulfate, mannitol, sucralose, crospovidone, talc under stirring and continue stirring till to get homogenous dispersion.
3. Add Lumacaftor to above solution of step 2 under stirring and continue stirring till to get homogenous dispersion.

### Stage-C:

Mix Ivacaftor dispersion of Stage-A and Lumacaftor dispersion of stage-B to get a uniform dispersion by either stirring or by homogenization.
4. Add orange Juice Flavor, peppermint flavor and glycerine to the dispersion of Stage-C under stirring and continue stirring till a homogenous dispersion is obtained.
5. Further, follow the steps 7 to 10 described in Example 1 to obtain Ivacaftor/Lumacaftor combination oral film.

### Example 4: Oral Film of Tezacaftor 25 mg

TABLE 4

| Sr. No | Ingredients | mg/film |
|---|---|---|
| 1 | Tezacaftor | 25 |
| 2 | Dichloro methane | q.s |
| 3 | Methanol | q.s |
| 4 | Polyvinyl pyrrolidone | 4 |
| 5 | Sodium Lauryl Sulfate | 0.20 |
| 6 | Mannitol | 6.80 |
| 7 | Sucralose | 2 |
| 8 | Glycerine | 10 |
| 9 | Orange Juice Flavor | 8 |
| 10 | Peppermint Flavor | 8 |
| 11 | Hydroxypropyl Methyl Cellulose | 52 |
| 12 | Purified Water | q.s |

| | |
|---|---|
| Film weight | 116 |

q.s: quantity sufficient

### Manufacturing Process:

### Stage-A: Preparation of Tezacaftor Dispersion

1. Add Dichloro methane to methanol under stirring and continue stirring for 5 min.
2. Add Polyvinylpyrrolidone to step 1 under stirring followed by sodium lauryl sulfate, mannitol, sucralose under stirring and continue stirring till to get homogenous solution.
3. Add Tezacaftor to above solution of step 2 under stirring and continue stirring till to get homogenous dispersion.

### Stage-B: Preparation of Polymer Solution

4. In a separate vessel, add Hydroxypropyl methylcellulose to purified water under stirring and continue stirring to get homogenous solution.

### Stage-C:

Mix Tezacaftor dispersion of Stage-A and polymer solution of Stage-B to get a uniform dispersion by either stirring or by homogenization.
5. Add orange juice flavor, peppermint flavor and glycerine to the dispersion of Stage-C under stirring and continue stirring till a homogenous dispersion is obtained.
6. Further, follow the steps 7 to 10 described in Example 1 to obtain Tezacaftor oral film.

### Example 5: Oral Film of Elexacaftor 50 mg

TABLE 5

| Sr. No | Ingredients | mg/film |
|---|---|---|
| 1 | Elexacaftor | 50 |
| 2 | Dichloro methane | q.s |
| 3 | Methanol | q.s |
| 4 | Methyl ethyl ketone | q.s |
| 5 | Polyvinyl pyrrolidone | 10 |
| 6 | Sodium Lauryl Sulfate | 0.50 |
| 7 | Mannitol | 15 |
| 8 | Sucralose | 2.50 |
| 9 | Glycerine | 7.50 |
| 10 | Orange Juice Flavor | 4 |
| 11 | Peppermint Flavor | 4 |
| 12 | Hydroxypropyl Methyl Cellulose | 115.0 |
| 13 | Purified Water | q.s |

| | |
|---|---|
| Film weight | 208.50 |

q.s: quantity sufficient

### Manufacturing Process:

Process described in example 4 can be followed to manufacture elexacaftor oral film, additionally, Methyl ethyl ketone is added to methanol along with dichloromethane in the process.

### Example 6: Oral Film of Ivacaftor 75 mg

TABLE 6

| | | Quantity per unit (mg) | | | | |
|---|---|---|---|---|---|---|
| S. | | Examples | | | | |
| no. | Ingredients | 6A | 6B | 6C | 6D | 6E |
| 1 | Ivacaftor | 75 | 75 | 75 | 75 | 75 |
| 2 | Hydroxypropyl | 10-20 | 10-20 | — | — | 18.38 |
| | methylcellulose | | | | | |
| | Acetate succinate | | | | | |
| 3 | Sodium Lauryl | 0.30-0.60 | 0.30-0.60 | 0.30-0.60 | 0.30-0.60 | 0.46 |
| | Sulfate | | | | | |
| 4 | Hydroxypropyl | 90 | 90 | 90-120 | 60-90 | 60-90 |
| | methylcellulose | | | | | |
| 5 | Copovidone | — | 30-45 | — | 30-45 | — |
| 6 | Crospovidone | 0-15 | 0-15 | 0-15 | 0-15 | 0-5 |
| 7 | Microcrystalline | 0-5 | 0-5 | 0-5 | 0-5 | 0-5 |
| | Cellulose | | | | | |
| 8 | Sucralose | 3 | 3 | 3 | 3 | 3 |
| 9 | Propylene glycol | 10-20 | 10-20 | 10-20 | 10-20 | — |
| 10 | Glycerine | — | — | — | — | 15 |
| 11 | Flavoring agent | 2 | 2 | 2 | 2 | 2 |
| 12 | Colorant | 0.02 | 0.02 | 0.02 | — | 0.02 |
| 13 | Dichloromethane | q.s | q.s | q.s | q.s | — |
| 14 | Methanol | q.s | q.s | q.s | q.s | — |
| 15 | Methyl Ethyl Ketone | q.s | q.s | q.s | q.s | q.s |
| 16 | Purified Water | q.s | q.s | q.s | q.s | q.s |

| | | | | | |
|---|---|---|---|---|---|
| Weight of Film | 190.32-230.62 | 220.32-275.62 | 180.32-240.62 | 180.3-255.6 | 173.86-213.86 |

### Manufacturing Process for Example 6A, 6B, 6C and 6D:

### Stage-A: Preparation of Polymer Dispersion

1. Transfer Methanol to manufacturing vessel and optionally add copovidone under stirring and continue stirring till a clear solution is obtained.
2. Add hydroxypropyl methylcellulose, sucralose, propylene glycol, optionally microcrystalline cellulose, optionally crospovidone, flavoring agent, colorant and dichloromethane to the solution of step 2 under stirring and continue stirring till homogenous dispersion is obtained.

### Stage-B: Preparation of Ivacaftor Dispersion

3. In a separate vessel add methyl ethyl ketone and purified water and mix.
4. Add Ivacaftor to step 3 and mix till clear solution is observed.
5. Add Hydroxypropyl methylcellulose and/or Hydroxypropyl methylcellulose acetate succinate to solution of step 4 under mixing followed by addition of sodium lauryl sulfate and continue stirring till homogenous dispersion is obtained.

### Stage C.

Mix Stage A and Stage B dispersions under stirring and continue stirring till a homogenous dispersion is obtained. Homogenize the dispersion using a homogenizer to obtain a homogenous dispersion.

Further, follow the steps 7 to 10 described in Example 1 to obtain Ivacaftor oral films.

### Manufacturing Process for Example 6E:

1. Preparation of Ivacaftor spray dried dispersion granules:
   a. Transfer Methyl ethyl ketone and Purified water into a vessel and mix.
   b. Further, add Hydroxypropylmethyl cellulose acetate succinate followed by Sodium Lauryl Sulfate followed by Ivacaftor into above solvent and continue stirring to get homogenous dispersion.
   c. The above dispersion is spray dried to obtain fine granules, which are vacuum oven dried to remove excess solvent.
2. In a separate vessel add Hydroxypropyl methylcellulose, purified water under stirring and add dried granules of step 1 and continue stirring to get a homogenous dispersion.
3. Add Sucralose, glycerin, optionally microcrystalline cellulose, optionally crospovidone, flavoring agent and colorant to the step 2 dispersion under stirring and continue stirring till a homogenous dispersion is obtained. Homogenize the dispersion using a homogenizer to obtain a homogenous dispersion.
4. Further, follow the steps 7 to 10 described in Example 1 to obtain Ivacaftor oral film.

### Example 7: Oral Film of Ivacaftor 62.5 mg/Lumacaftor 50 mg

TABLE 7

| | | Quantity per unit (mg) | | | | |
|---|---|---|---|---|---|---|
| S. | | Example | | | | |
| No. | Ingredients | 7A | 7B | 7C | 7D | 7E |
| 1 | Lumacaftor | 50 | 50 | 50 | 50 | 50 |
| 2 | Ivacaftor | 62.50 | 62.50 | 62.50 | 62.50 | 62.50 |
| 3 | Hydroxypropyl | 10-20 | 10-20 | — | — | 18.38 |
| | methylcellulose | | | | | |
| | Acetate succinate | | | | | |
| 4 | Sodium Lauryl Sulfate | 0.30-0.60 | 0.30-0.60 | 0.30-0.60 | 0.30-0.60 | 0.46 |
| 5 | Hydroxypropyl | 120 | 120 | 90-120 | 60-90 | 90-120 |
| | methylcellulose | | | | | |
| 6 | Copovidone | — | 30-45 | — | 30-45 | — |
| 7 | Crospovidone | 0-15 | 0-15 | 0-15 | 0-15 | 0-5 |
| 8 | Microcrystalline | 0-5 | 0-5 | 0-5 | 0-5 | 0-5 |
| | Cellulose | | | | | |
| 9 | Sucralose | 3 | 3 | 3 | 3 | 3 |
| 10 | Propylene glycol | 10-20 | 10-20 | 10-20 | 10-20 | — |
| 11 | Glycerine | — | — | — | — | 10-20 |
| 12 | Flavoring agent | 2 | 2 | 2 | 2 | 2 |
| 13 | Colorant | 0.02 | 0.02 | 0.02 | 0.02 | 0.02 |
| 14 | Dichloromethane | q.s | q.s | q.s | q.s | q.s |
| 15 | Methanol | q.s | q.s | q.s | q.s | q.s |
| 16 | Methyl Ethyl Ketone | q.s | q.s | q.s | q.s | q.s |
| 17 | Purified Water | q.s | q.s | q.s | q.s | q.s |

| | | | | | |
|---|---|---|---|---|---|
| Weight of Film | 257.82-298.12 | 287.82-343.12 | 217.82-278.12 | 217.82-293.12 | 236.36-286.36 |

q.s: quantity sufficient

### Manufacturing Process for Example 7A, 7B, 7C and 7D:

### Stage-A: Preparation of Lumacaftor Dispersion

1. Transfer Methanol to Manufacturing vessel and add Lumacaftor under stirring and continue stirring till a clear solution is obtained.
2. Add Hydroxypropyl methylcellulose, optionally copovidone, sucralose, propylene glycol, optionally crospovidone, optionally microcrystalline cellulose, flavoring agent, coloring agent and dichloromethane to the dispersion of step 1 under stirring and continue stirring till a homogenous dispersion is obtained.

### Stage-B: Preparation of Ivacaftor Dispersion

Ivacaftor polymer dispersion can be obtained according to the process described in stage B of example 6A, 6B, 6C and 6D.

### Stage C.

Mix Stage A and Stage-B dispersions under stirring and continue stirring till a homogenous dispersion is obtained. Homogenize the dispersion using a homogenizer to obtain a homogenous dispersion.

Further, follow the steps 7 to 10 described in Example 1 to obtain Ivacaftor/Lumacaftor combination oral film.

### Manufacturing Process for Example 7E:

### Stage-A Preparation of Ivacaftor Spray Dried Dispersion Granules:

Ivacaftor spray dried dispersion granules are obtained according to the process described in example 6E.

### Stage-B Preparation of Lumacaftor Dispersion:

In a separate vessel transfer water and add Hydroxypropylmethyl cellulose under stirring and further add Lumacaftor under stirring and continue stirring to get a homogenous dispersion.

### Stage-C:

1. Mix Stage A and Stage B under stirring and continue stirring to get a homogenous dispersion.
2. Add Sucralose, glycerine, optionally microcrystalline cellulose, optionally crospovidone, flavoring agent and colorant to step 1 dispersion under stirring and continue stirring to get homogenous dispersion.
3. Further follow the steps 7 to 10 described in Example 1 to obtain oral film of Ivacaftor/Lumacaftor combination.

### Example 8: Oral Film of Ivacaftor 75 mg/Tezacaftor 50 mg

TABLE 8

| | | Quantity per unit (mg) | | | | |
|---|---|---|---|---|---|---|
| S. | | Example | | | | |
| No. | Ingredients | 8A | 8B | 8C | 8D | 8E |
| 1 | Tezacaftor | 50 | 50 | 50 | 50 | 50 |
| 2 | Ivacaftor | 75 | 75 | 75 | 75 | 75 |
| 3 | Hydroxypropyl | 10-20 | 10-20 | — | — | 18.38 |
| | methylcellulose | | | | | |
| | Acetate succinate | | | | | |
| 4 | Sodium Lauryl | 0.30-0.60 | 0.30-0.60 | 0.30-0.60 | 0.30-0.60 | 0.46 |
| | Sulfate | | | | | |
| 5 | Hydroxypropyl | 120 | 120 | 90-120 | 60-90 | 60-90 |
| | methylcellulose | | | | | |
| 6 | Copovidone | — | 30-45 | — | 30-45 | — |
| 7 | Crospovidone | 0-15 | 0-15 | 0-15 | 0-15 | 0-5 |
| 8 | Microcrystalline | 0-5 | 0-5 | 0-5 | 0-5 | 0-5 |
| | Cellulose | | | | | |
| 9 | Sucralose | 3 | 3 | 3 | 3 | 3 |
| 10 | Propylene glycol | 10-20 | 10-20 | 10-20 | 10-20 | — |
| 11 | Glycerin | — | — | — | — | 10-20 |
| 12 | Flavoring agent | 2 | 2 | 2 | 2 | 2 |
| 13 | Colorant | 0.02 | 0.02 | 0.02 | 0.02 | 0.02 |
| 14 | Dichloromethane | q.s | q.s | q.s | q.s | q.s |
| 15 | Methanol | q.s | q.s | q.s | q.s | q.s |
| 16 | Methyl Ethyl Ketone | q.s | q.s | q.s | q.s | g.s |
| 17 | Purified Water | q.s | q.s | q.s | q.s | q.s |

| | | | | | |
|---|---|---|---|---|---|
| Weight of Film | 270.32-310.62 | 300.32-355.62 | 230.32-290.62 | 230.32-302.62 | 218.86-268.86 |

q.s: quantity sufficient

### Manufacturing Process for Example 8A, 8B, 8C and 8D:

### Stage-A: Preparation of Tezacaftor Dispersion

1. Transfer methanol to manufacturing vessel and add Tezacaftor under stirring and continue stirring till to get uniform dispersion.
2. Add Hydroxypropyl methylcellulose, sucralose, propylene glycol, optionally copovidone, optionally crospovidone, optionally microcrystalline cellulose, flavoring agent, colorant, and Dichloromethane to the dispersion of step 1 under stirring and continue stirring till to get homogenous dispersion.

### Stage-B: Preparation of Ivacaftor Dispersion

Ivacaftor dispersion is prepared according to the process described in example 6A, 6B, 6C and 6D.

### Stage-C:

Mix Stage A and Stage B under stirring and continue stirring till to get a homogenous dispersion.

Further follow the steps 7 to 10 described in Example 1 to obtain oral film of Ivacaftor/Tezacaftor.

### Manufacturing Process for Example 8E:

### Stage-A Preparation of Ivacaftor Spray Dried Dispersion Granules:

Ivacaftor spray dried dispersion granules are prepared according to the process described in Example 6E.

### Stage-B Preparation of Tezacaftor Spray Dried Dispersion Granules:

1. Transfer Methanol and Dichloromethane into a vessel and mix.
2. Add part quantity of Hydroxypropyl methylcellulose followed by Tezacaftor to step 1 under stirring and continue stirring to get a homogenous dispersion.
3. Dispersion of step 2 is spray dried to obtain fine granules, which are vacuum oven dried to remove excess solvent.

### Stage-C

In a separate vessel, add purified water and remaining quantity of Hydroxypropyl methylcellulose under stirring. Further, Stage A and Stage B dried granules are added under stirring and continue stirring to get a homogenous dispersion followed by sucralose, glycerin, optionally microcrystalline cellulose, optionally crospovidone, flavoring agent and colorant under stirring and continued stirring till a homogenous dispersion is obtained.

Further follow the steps 7 to 10 described in Example 1 to obtain oral film of Ivacaftor/Tezacaftor combination.

1. An oral film of cystic fibrosis transmembrane conductance regulator (CFTR) modulator(s) and its pharmaceutically acceptable salts thereof.

2. The oral film as claimed in claim 1, wherein the oral film comprises:
i. cystic fibrosis transmembrane conductance regulator (CFTR) modulator(s) and its pharmaceutically acceptable salt thereof;
ii. at least one film forming polymer;
iii. plasticizer;
iv. optionally carrier(s); and
v. other pharmaceutically acceptable excipients.

3. The oral film as claimed in claim 1, wherein the oral film comprises:
i. Solid dispersion(s) of cystic fibrosis transmembrane conductance regulator (CFTR) modulator(s) and its pharmaceutically acceptable salt thereof;
ii. optionally another cystic fibrosis transmembrane conductance regulator (CFTR) modulator(s);
iii. at least one film forming polymer;
iv. plasticizer; and
v. other pharmaceutically acceptable excipients.

4. The oral film as claimed in claim 1, wherein the cystic fibrosis transmembrane conductance regulator (CFTR) modulator is selected from Ivacaftor, Lumacaftor, Tezacaftor, Elexacaftor or combination of Ivacaftor/Lumacaftor, Ivacaftor/tezacaftor, Ivacaftor/tezacaftor/elexacaftor, or any other combinations and their pharmaceutically acceptable salts thereof.

5. The oral film as claimed in claim 2, wherein the film forming polymer is selected from the group comprising of hydrophilic polymer(s) selected from hydroxypropylmethyl cellulose, hydroxyethyl cellulose, hydroxypropyl cellulose, sodium carboxy methylcellulose, hydroxypropylmethylcellulose acetate succinate, polyvinyl pyrrolidone or povidone, copovidone, polydextrose, polyvinyl alcohol, polyvinyl acetate, polyethylene oxide, pullulan, sodium alginate, propylene glycol alginate, polyacrylic acid, copolymers of acrylic acid, carboxyvinyl copolymers, modified starch, gelatin, pectin, hydroxypropylethylcellulose, polyoxyethylene stearates, poly-epsilon caprolactone, polyglycolized glycerides, cyclodextrins, carrageenan, galactomannans, polymerized rosin and combinations thereof and/or hydrophobic polymer(s) selected from ethyl cellulose, cellulose acetate, butyl cellulose, polymethacrylates, shellac, stearic acid, glyceryl behenate, palmitic acid and combinations thereof.

6. The oral film as claimed in claim 2, wherein the plasticizer is selected from the group comprising of polyethylene glycol, propylene glycol, polyethylene-propylene glycol, glycerol, glycerol monoacetate, diacetate or triacetate, triacetin, polysorbate, cetyl alcohol, sorbitol, sodium diethylsulfosuccinate, triethyl citrate, tributyl citrate, phthalate derivatives such as dimethyl, diethyl and dibutyl phthalate and combinations thereof.

7. The oral film as claimed in claim 2, wherein the carrier is selected from the group comprising of povidone, copovidone, polyvinyl alcohol, cellulose derivatives including hydroxypropylmethylcellulose acetate succinate, polyvinyl caprolactam-polyvinyl acetate-polyethylene glycol graft co-polymer, hydroxypropyl methylcellulose, hydroxypropyl cellulose, sodium carboxymethylcellulose, hydroxyethyl cellulose, hydroxypropylmethylcellulose phthalate, polymethacrylic polymers, polyethylene glycol, polyoxyethylene oxide, poloxamers, polyoxyethylene stearates, poly-epsilon caprolactone, polyglycolized glycerides, pectins, cyclodextrins, galactomannans, dextrin, mannitol, alginates, carragenan, xanthan gum and combinations thereof.

8. The oral film as claimed in claim 2, wherein other pharmaceutically acceptable excipients are selected from the group comprising of suspending/thickening agents, disintegrating agents, fillers/bulking agents, stabilizers, surfactants, sweetening agents, taste masking agents, anti-foaming agents, flavoring agents, coloring agents and combinations thereof.

9. The oral film as claimed in claim 1, wherein the composition comprises:
i. from about 5% to about 75% by weight of cystic fibrosis transmembrane conductance regulator (CFTR) modulator or combinations of cystic fibrosis transmembrane conductance regulator (CFTR) modulators and its pharmaceutically acceptable salts;
ii. from about 1% to about 60% by weight of at least one film forming polymer;
iii. from about 1% to about 15% by weight of plasticizer;
iv. from 0% to about 50% by weight of carrier(s); and
v. other pharmaceutically acceptable excipients.
wherein the percentage by weight is relative to the total weight of the composition.

10. The oral film as claimed in claim 1, wherein the composition comprises:
i. from about 20% to about 95% by weight of solid dispersion(s) of cystic fibrosis transmembrane conductance regulator (CFTR) modulator(s) and its pharmaceutically acceptable salts;
ii. from about 1% to about 60% by weight of at least one film forming polymer;
iii. from about 1% to about 15% by weight of plasticizer; and
iv. other pharmaceutically acceptable excipients.
wherein the percentage by weight is relative to the total weight of the composition.
